(19) United States
(12) Patent Application Publication
(10) Pub. No.: US 2024/0238169 A1
(43) Pub. Date: Jul. 18, 2024

(54) POWDERY CLEANSING COMPOSITION

(71) Applicant: BEIERSDORF AG, Hamburg (DE)

(72) Inventors: Eefje KOOP, Norderstedt (DE); Rene HEINZ, Quickborn (DE); Wiebke SCHLUESCHE, Halstenbek (DE); Cecile RATSCHOW, Hamburg (DE); Koushik SREEDHAR, Hamburg (DE)

(21) Appl. No.: 18/559,842

(22) PCT Filed: May 10, 2022
(86) PCT No.: PCT/EP2022/062640

§ 371 Date: Nov. 9, 2023

(30) Foreign Application Priority Data
Jun. 22, 2021 (DE) 10 2021 206 396.9

(51) Int. Cl. A61K 8/02; A61Q 19/10

(52) U.S. Cl. CPC A61K 8/022; A61Q 19/10; A61K 2800/31; A61K 2800/596; A61K 2800/87; A61K 2800/882

(57) ABSTRACT

The present invention belongs to the cosmetic field and relates to a substantially anhydrous cosmetic powder based cleansing formulations characterised by contents of sodium coco sulfate and/or disodium lauryl sulfosuccinate, and sodium benzoate and hydroxyacetophenone.

The present invention belongs to the cosmetic field and relates to a powder based cleansing formulation.

A beautiful and attractive appearance is a desire for many people. One typical sign of such an appearance is a healthy and smooth looking skin. Therefore, to take care on the skin, it is for many people a daily routine to apply cosmetic products such as cleansing and moisturizing products.

Conventional cleansing formulations are mostly sold in form of a liquid, aqueous formulation. Analyzing the ingredients of such a formulation it can be noted that the formulation comprises a significant amount of water. Typical examples of shower gels comprising 20% by weight to 95% by weight of water are disclosed in WO1994010975A1, WO1993021900A1 and WO2002005758A2.

Accordingly, significant amounts of water are shipped from the supplier to the stores and further to the customer, to provide the customer with the cleansing formulation of his or her choice. Taking the fact into consideration that most customer's homes are connected to the public water supply, it seems environmentally not sensible to ship water to a destination which has basically unlimited access to water. Thus, by omitting water in the formulations and shipping a concentrate either in liquid or solid form transport cost and transport-related emissions of greenhouse gases may be reduced.

Novel concepts involve e.g. the shipment of concentrates and a mixing with water at the point of sale. Other approaches use solid cleansing compositions in form of a bar. The latter named formulations are applied by the customer such as a soap bar, e.g. by rubbing the bar over the body. However, the use of a solid bar to apply wash active ingredients to the human body is often not desirable for customers who got use to apply cleansing formulations in liquid form.

Accordingly, there is a general need to provide cleansing formulations which transport and use has a minor environmental impact, e.g. releasing fewer greenhouse gases upon transport.

This objective can be met by providing the customer with a substantially anhydrous powder-based cleansing formulation, which is dissolvable in water. Accordingly, the customer can buy the powder-based formulation in a shop. At home the customer simply needs to add the powder to a predetermined amount of tap water, to allow an aqueous cleansing formulation to be formed.

Thereby, it is preferred, if the powder-based formulation is encapsulated in a way, that customer cannot get in contact with the powder. This may be achieved by enclosing the powder-based formulation in a water-soluble pouch. The most favored version is to use polyvinyl alcohol films which fully deserve in water and allow a release of the concentrated powder-based formulation.

Although this concept reads rather simple, the person skilled in art faces several problems in the development of such a product as described above, which will be outlined in the following:

One aspect is that the quality of the tab water used to prepare the cleansing formulation may vary depending on where the customer sources the water. Accordingly, it is essential to take some measures which ensure that the cleansing formulation does not turn into a bacterial source. Accordingly, in a first step, it is essential that some agents are contained, which allow the bacterial concentration to be controlled. In a second step, the formulations often contain a pH-buffer system ensuring that after mixing with different qualities of tap water a specific target pH in the range between 4 and 5 is always obtained.

Typical agents to avoid and/or reduce the bacterial concentration in cosmetic compositions are e.g. sodium benzoate, hydroxyacetophenone, hexamidine diisethionate, chlorphenesin and DMDM hydantoin. The buffering system comprises usually sodium citrate and citric acid.

Although, in the most cases, the cleansing formulations formed by dissolving the powder base formulation in tab water are free from flocculation and can be described as a clear and/or transparent, it is often observed that those formulations show instabilities if stored at particular temperatures for several days. For example, it was found that the storage at temperatures below 8° C., in particular at −10° C., for a week may lead to the formation of flakes or turbid formulations. Without being bound by the theory, it is believed that this phenomenon is the result of an interaction of the agent to reduce the bacterial concentration which may interact with other ingredients of the formulation. In some cases, this might be that buffering system, which ensures a low pH value. Accordingly, it is not possible to provide a cleansing composition to be dissolved in tab water having sufficient preservatives included to ensure no bacterial growth, which has a pH value after dissolution in water of 5 or less and which is table not showing flocculation effects, such as forming a turbid formulation within 7 days at any temperature between 8 and minus 10° C.

The described problems result in the effect that consumers are often disappointed when comparing the home-made cleansing product to conventional products supplied including an aqueous phase.

Accordingly, it is the objective of the present invention to address at least some of the problems described above such that the consumer is supplied with a powder based formulation which he can use to prepare an aqueous cleansing formulation at home.

It was surprisingly found by the applicant that the problems described above can be addressed or at least reduced by the present invention.

The present invention is a substantially anhydrous cosmetic powder composition comprising
a) sodium coco sulfate and/or disodium lauryl sulfosuccinate, and
b) sodium benzoate, and
c) hydroxyacetophenone.

According to the invention the term “powder composition” is understood as a composition comprising less than 10% by weight, preferably less than 8% by weight, more preferably less than 6% by weight and most preferably less than 5.9% by weight of ingredients which are liquid under normal conditions, whereby the % by weight refer to the total weight of the composition. The rest of the composition consist solely of ingredients which are solid under normal condition.

According to the invention the term “anhydrous composition” is understood as a composition comprising less than 9.9% by weight, preferably less than 7.9% by weight and most preferably less than 5.9% by weight of water, whereby the % by weight refer to the total weight of the composition.

Unless otherwise stated, all tests and measurements were performed under “normal conditions”. The term “normal conditions” refers to 20° C., 1013 hPa and a relative humidity of 50%.

All temperatures disclosed refer, unless otherwise stated, to a pressure of 1013 hPa.

If percentages by weight (% by weight) are given below without reference to a particular composition or specific mixture, these figures always refer to the total weight of the composition.

If ratios of certain components are disclosed without reference in the following description, these ratios always refer to weight ratios of the components.

If ranges for the weight of certain ingredients are disclosed, the disclosure includes not only the start and end values for the indicated ranges but also the individual values within the ranges in steps of 0.01% by weight.

According to the invention it is preferred if the total quantity of the sodium coco sulfate and disodium lauryl sulfosuccinate is in the range from 25 to 65% by weight, more preferably 30 to 55% by weight and most preferably 32 to 52% by weight calculated to the total weight of the composition.

It is preferred according to the invention if sodium coco sulfate is contained in the composition in a total quantity from 10 to 28% by weight, more preferably 12 to 26% by weight and most preferably 14 to 24% by weight calculated to the total weight of the composition.

It is preferred according to the invention if disodium lauryl sulfosuccinate is contained in the composition in a total quantity from 10 to 26% by weight, more preferably 12 to 24% by weight and most preferably 14 to 20% by weight calculated to the total weight of the composition.

It is preferred if the ratio by weight between the quantity of sodium coco sulfate and disodium lauryl sulfosuccinate is in the range from 1:0.2 to 1:2, more preferably from 1:0.3 to 1:1.5 and most preferably from 1:0.8 to 1:1.2.

In some embodiments of the invention, it is preferred if the composition comprises one surfactant selected from the group consisting of sodium myristoyl glutamate, sodium lauroyl glutamate, sodium stearoyl glutamate and sodium cocoyl glutamate. The total quantity of the surfactants selected from the group consisting of sodium myristoyl glutamate, sodium lauroyl glutamate, sodium stearoyl glutamate and sodium cocoyl glutamate is in the range from 5 to 33% by weight, more preferably from 10 to 27% by weight, still more preferably from 15 to 25% by weight and most preferably from 20 to 24% by weight, calculated to the total weight of the composition.

Other embodiments of the invention are preferably characterized in that the total quantity of the surfactants selected from the group consisting of sodium myristoyl glutamate, sodium lauroyl glutamate, sodium stearoyl glutamate and sodium cocoyl glutamate is in the range from 0% to 5% by weight, more preferably 0% to 4% by weight, more preferably from 0% to 3% by weight, more preferably from 0% to 2% by weight, more preferably from 0% to 1% by weight, more preferably from 0% to 0.5% by weight, more preferably from 0% to 0.25% by weight, more preferably from 0% to 0.1% by weight and most preferably in total quantity of 0% by weight, based on the total weight of the composition.

In some embodiments of the invention, it is preferred if the compositions comprises sodium methyl cocoyl taurate. It is preferred according to the invention if sodium methyl cocoyl taurate is contained in the composition in a total quantity from 6 to 26% by weight, more preferably 8 to 20% by weight and most preferably 10 to 18% by weight calculated to the total weight of the composition. Thereby, it is preferred if the ratio by weight between the quantity of sodium coco sulfate and sodium methyl cocoyl taurate is in the range from 1:0.2 to 1:2, more preferably from 1:0.3 to 1:1.5 and most preferably from 1:0.8 to 1:1.2.

Other embodiments are characterized in that sodium methyl cocoyl taurate is not contained in the composition. That means the total quantity of sodium methyl cocoyl taurate is 0% by weight, calculated to the total weight of the composition.

According to the present invention it is preferred if the composition comprises further surfactants in a total quantity from 0% to 5% by weight, more preferably 0% to 4% by weight, more preferably from 0% to 3% by weight, more preferably from 0% to 2% by weight, more preferably from 0% to 1% by weight, more preferably from 0% to 0.5% by weight, more preferably from 0% to 0.25% by weight, more preferably from 0% to 0.1% by weight and most preferably in total quantity of 0% by weight, calculated to the total weight of the composition.

Compositions which are according to the present invention are further characterized in that they comprise sodium benzoate and hydroxyacetophenone.

If sodium benzoate is contained, the total quantity of sodium benzoate is preferably in the range from 4 to 30% by weight, more preferably 8 to 28% by weight, more preferably 10 to 25% by weight and most preferably 15 to 23% by weight, calculated to the total weight of the composition.

If hydroxyacetophenone is contained, the total quantity of hydroxyacetophenone is preferably in the range from 2% to 15% by weight, more preferably 3% to 12% by weight and most preferably 6% to 10% by weight, calculated to the total weight of the composition.

Further, it is preferred if the ratio by weight between sodium benzoate and all contained surfactants is in the range from 1:1.5 to 1:8, more preferably from 1:1.8 to 1:6 and most preferably from 1:1.9 to 1:5.

For the case, that both sodium benzoate and hydroxyacetophenone are contained, it is preferred if the ratio by weight between sodium benzoate and hydroxyacetophenone is in the range from 5:1 to 1:1, more preferred from 4:1 to 2:1 and most preferred from 3:1 to 2.5:1.

Compositions which are preferred according to the present invention are further characterized in that they comprise citric acid and/or sodium citrate. If citric acid and/or sodium citrate are contained it is preferred if the total quantity of both is in the range from 10 to 35% by weight, more preferably 15 to 30% by weight and most preferably 20 to 26% by weight, calculated to the total weight of the composition.

It is preferred if both sodium citrate and citric acid are contained. For the case that both sodium citrate and citric acid are contained, it is preferred if the ratio by weight between sodium citrate and citric acid is from 1:2 to 1:10, more preferred from 1:4 to 1:8 and most preferred from 1:6 to 1:7.5.

Other embodiments of the invention are characterized in that the ratio by weight between sodium citrate and citric acid is adjusted such that the composition has a pH in the range between 3.2 and 5, more preferably in the range between 3.5 and 4.9 and most preferably between 4.2 to 4.8 for the case the composition is dissolved in water in a ratio of 1 part composition and 20 parts water.

Further preferred compositions according to the invention are characterized in that the compositions do not comprise DMDM hydantoin, chlorphenesin, iodpropylbutylcarbamate and/or hexamidine diisethionate.

Other preferred compositions according to the invention may comprise DMDM hydantoin, chlorphenesin, iodpropylbutylcarbamate and/or hexamidine diisethionate.

If DMDM hydantoin is contained, the total quantity of DMDM hydantoin is preferable in the range from 0.5 to 12% by weight, calculated to the total weight of the composition.

If chlorphenesin is contained, the total quantity of chlorphenesin is preferable in the range from 0.5 to 13% by weight, calculated to the total weight of the composition.

If iodpropylbutylcarbamate is contained, the total quantity of iodpropylbutylcarbamate is preferable in the range from 0.1 to 13% by weight, calculated to the total weight of the composition.

If hexamidine diisethionate is contained, the total quantity of hexamidine diisethionate is preferable in the range from 0.1 to 5% by weight, calculated to the total weight of the composition.

The composition of the invention is preferably characterized in that once it is diluted in water the obtained solution has a pH value in the range from 3.2 to 5, more preferably in the range from 3.5 to 4.9 and most preferably from 4.2 to 4.8.

The powder composition according to the invention is preferably mixed into water to form an aqueous cosmetic cleansing formulation. Thereby it is preferred if the ratio by weight between water and the powder composition according to the invention is in the range from 9:1 to 99:1, more preferably from 92:8 to 98:2 and most preferred from 94:6 to 97:3.

Accordingly, another aspect of the invention is a method to provide an aqueous cosmetic cleansing composition comprising the steps of providing the substantially anhydrous cosmetic powder composition and adding the composition to water, whereby it is preferred if the ratio by weight between water and the powder composition is in the range from 9:1 to 99:1, more preferably from 92:8 to 98:2 and most preferred from 94:6 to 97:3.

According to the invention it is further advantages, if the powder composition of the invention is packaged into an enclosing packaging. In this way the customer can open the packaging in order to add the powder of the invention to water.

As such, the invention is also a cosmetic product comprising a packaging and the cosmetic powder composition of the invention, whereby the packaging encloses the cosmetic powder composition of the invention.

In a first embodiment the packaging of the cosmetic product comprises at least one section which consists of a water-soluble film and at least one section which consist of a material which is not water soluble. A preferred example of such a packaging is a cap for a bottle, wherein the cap is designed in a way such that once the cap is attached to a bottle, the section with a water soluble film is located inside the bottle. By this means the cap can be used on a bottle filled with tap water. Once the cap is attached the bottle and water is in contact with the water-soluble film, the cosmetic powder composition is released into the inside of the bottle to form the cosmetic cleansing composition to be used by the customer.

It is preferred if the cap is designed such it can be removed from the bottle to either have the possibility to take out the composition from the bottle for use, or to allow for the exchange of caps such that the bottle can be reused. By this means transport costs can be reduced as only the cap is sold to the customer, while the bottle can be reused.

It is further preferred if the cap has a thread to be removable attached to the bottle. Furthermore, it is preferred if the cap enclosing the powder composition is designed such that the cap has a closable opening oriented in the cap such that for the case the cap is attached to a bottle the content of the bottle can be removed. Furthermore, the cap is preferably characterized in that it has a removable seal which is attached to cap in a way that it allows no water to get into contact with the water soluble film. In this way, it is avoided that the cosmetic powder composition is accidentally released if the packaging becomes wet upon transport or at home. The seal can be removed by hand or, in preferred embodiments, the seal is attached to the cap such it automatically breaks one the cap is attached to a matching bottle.

In another embodiment the packaging is also a cap such as in the embodiment described above. However, this embodiments differs in that the powder composition is enclosed in the cap by a film which is ripped if the cap is attached to the bottle, such the powder composition is automatically released into the inside of the bottle.

In still another embodiment, cosmetic product is characterized in that the powder composition of the invention is enclosed by the packaging, whereby the packaging consists of a film, which is water-soluble. Accordingly, the cosmetic product of this embodiment may be put into a bottle filled with water, whereby the film breaks and releases the powder composition.

According to the invention the term “water-soluble film” is understood as a film, which breaks once the film is surrounded by water.

It is preferred if the water-soluble film described in the embodiments above comprises or consists of polyvinyl alcohol, gelantine and/or alginates, whereby polyvinyl alcohol is preferred.

In still another embodiment of the invention it is preferred if the composition is enclosed in a paper pouch. In this case the paper pouch enclosing the composition may be put into a bottle filled with water. The paper will break and/or dissolve upon the contact with water, allowing to composition to be released. Alternatively, the customer may rip the paper before adding solely the composition of the invention to the water.

### EXAMPLES

The following examples should illustrate the present invention, without intending to limit the invention to these examples. The numerical values in the examples are percentages by weight, based on the total weight of the preparations. In the case the formulations do not sum up to 100% by weight, the content is adjusted by the water content such that the formulations sums up 100% by weight.

In the table below the formulations with the designation Com. are comparative examples, while all examples with the designation Ex. are examples according to the invention.

| Ingrediens | Ex. 1 | Com. 1 | Com. 2 | Com. 3 |
|---|---|---|---|---|
| Sodium Lauryl | 17.86 | 22.73 | 22.06 | 23.51 |
| Glutamate | | | | |
| Sodium Methyl | 0.00 | 0.00 | 0.00 | 0.00 |
| Lauroyl Taurate | | | | |
| Sodium Coco Sulfate | 17.32 | 22.05 | 21.40 | 22.81 |
| Disodium Lauryl | 16.96 | 21.59 | 20.96 | 22.34 |
| Sulfosuccinate | | | | |
| Citric Acid | 23.81 | 15.15 | 14.71 | 15.67 |
| Sodium Citrate | 3.57 | 10.61 | 10.29 | 10.97 |
| Sodium Benzoate | 10.71 | 0.00 | 0.00 | 0.00 |
| Hydroxyaceto- | 8.33 | 0.00 | 0.00 | 0.00 |
| phenone | | | | |
| Hexamidine | 0.00 | 0.00 | 0.00 | 2.82 |
| Diisethionate | | | | |
| Chlorphenesin | 0.00 | 0.00 | 8.82 | 0.00 |
| DMDM Hydantoin + | 0.00 | 6.06 | 0.00 | 0.00 |
| IPBC | | | | |
| Aqua | Ad 100 | Ad 100 | Ad 100 | Ad 100 |
| Mixed with water in | 1:22.8 | 1:29.3 | 1:28.4 | 1:30.3 |
| a ratio by weight | | | | |
| composition:water | | | | |
| Solibility in water | Yes, | Yes | Yes | Yes |
| (20° C.) | trans- | trans- | trans- | trans- |
| | parent | parent | parent | parent |
| Observation after 14 | trans- | floc- | floc- | floc- |
| days at 6° C. | parent | culates | culates | culates |
| pH after dissolution | 4.2 | 4.9 | 4.8 | 4.8 |
| in water | | | | |

| Ingrediens | Ex. 2 | Com. 4 | Com. 5 | Com. 6 |
|---|---|---|---|---|
| Sodium Methyl | 11.84 | 17.05 | 16.54 | 17.63 |
| Lauroyl Taurate | | | | |
| Sodium Coco Sulfate | 15.32 | 22.05 | 21.40 | 22.81 |
| Disodium Lauryl | 15.00 | 21.59 | 20.96 | 22.34 |
| Sulfosuccinate | | | | |
| Citric Acid | 21.05 | 15.15 | 14.71 | 15.67 |
| Sodium Citrate | 3.16 | 10.61 | 10.29 | 10.97 |
| Sodium Benzoate | 21.05 | 0.00 | 0.00 | 0.00 |
| Hydroxyacetophenone | 7.37 | 0.00 | 0.00 | 0.00 |
| Hexamidine | 0.00 | 0.00 | 0.00 | 2.82 |
| Diisethionate | | | | |
| Chlorphenesin | 0.00 | 0.00 | 8.82 | 0.00 |
| DMDM Hydantoin + | 0.00 | 6.06 | 0.00 | 0.00 |
| IPBC | | | | |
| Aqua | Ad 100 | Ad 100 | Ad 100 | Ad 100 |
| Mixed with water in a | 1:20.1 | 1:29.3 | 1:28.4 | 1:30.3 |
| ratio by weight | | | | |
| composition:water | | | | |
| Solibility in water | Yes, | Yes, | Yes, | Yes, |
| (20° C.) | trans- | trans- | trans- | trans- |
| | parent | parent | parent | parent |
| Observation after 7 | trans- | floc- | turbid | floc- |
| days at −10° C. | parent | culates | | culates |
| pH after dissolution | 4.1 | 4.6 | 4.6 | 4.5 |
| in water | | | | |

In addition, the inventive Examples were sealed in a polyvinylalcohol film and added to a water phase in the mixing ratios as described in the two tables above. It was observed that the sealed pouches released the powder and cosmetic cleansing compositions were formed. It was found that the film dissolved fully in the aqueous composition leaving no solid residues. The film had no effects on the flocculation or turbidity of the formed compositions upon storage.

The following example formulations are intended to illustrate the invention further. The compositions were mixed with water in a ratio composition to water of 1:26 and 1:21.

| Ingrediens | Ex. 3 | Ex. 4 | Ex. 5 | Ex. 6 | Ex. 7 | Ex. 8 |
|---|---|---|---|---|---|---|
| Sodium Lauryl | 15.48 | 0.00 | 0.00 | 0.00 | 0.00 | 0.00 |
| Glutamate | | | | | | |
| Sodium Methyl | 0.00 | 8.78 | 10.61 | 13.39 | 11.98 | 0.00 |
| Lauroyl Taurate | | | | | | |
| Sodium Coco | 18.90 | 16.53 | 18.90 | 17.32 | 18.10 | 25.40 |
| Sulfate | | | | | | |
| Disodium Lauryl | 18.51 | 16.19 | 18.51 | 16.96 | 16.75 | 23.54 |
| Sulfosuccinate | | | | | | |
| Citric Acid | 25.97 | 22.73 | 25.97 | 23.81 | 22.51 | 25.17 |
| Sodium Citrate | 3.90 | 3.41 | 3.90 | 3.57 | 3.75 | 3.28 |
| Sodium Benzoate | 11.69 | 22.73 | 11.69 | 10.71 | 18.50 | 15.31 |
| Hydroxyaceto- | 4.00 | 4.00 | 4.00 | 8.33 | 7.39 | 6.37 |
| phenone | | | | | | |
| Aqua | Ad | Ad | Ad | Ad | Ad | Ad |
| | 100 | 100 | 100 | 100 | 100 | 100 |

1.-19. (canceled)

20. A cosmetic composition, wherein the composition is a substantially anhydrous powder composition comprising
(a) sodium coco sulfate and/or disodium lauryl sulfosuccinate,
(b) sodium benzoate, and
(c) hydroxyacetophenone.

21. The composition of claim 20, wherein the composition comprises from 25% to 65% by weight of (a), based on a total weight of the composition.

22. The composition of claim 20, wherein the composition comprises from 10% to 28% by weight of (b), based on a total weight of the composition.

23. The composition of claim 20, wherein the composition comprises from 10% to 26% by weight of disodium lauryl sulfosuccinate, based on a total weight of the composition.

24. The composition of claim 20, wherein the composition comprises sodium coco sulfate and disodium lauryl sulfosuccinate in a weight ratio of from 1:0.2 to 1:2.

25. The composition of claim 20, wherein the composition further comprises sodium methyl cocoyl taurate.

26. The composition of claim 25, wherein the composition comprises sodium coco sulfate and sodium methyl cocoyl taurate in a weight ratio of from 1:0.2 to 1:2.

27. The composition of claim 20, wherein the composition comprises at least one surfactant selected from sodium myristoyl glutamate, sodium lauroyl glutamate, sodium stearoyl glutamate and sodium cocoyl glutamate.

28. The composition of claim 27, wherein the composition comprises the at least one surfactant in a concentration of from 5% to 33% by weight, based on a total weight of the composition.

29. The composition of claim 28, wherein the composition comprises further surfactants in a total quantity from 0% to 5% by weight, based on a total weight of the composition.

30. The composition of claim 20, wherein the composition comprises from 4% to 30% by weight of (b), based on a total weight of the composition.

31. The composition of claim 20, wherein a ratio by weight of (b) and all surfactants present in the composition is from 1:1.5 to 1:8.

32. The composition of claim 20, wherein the composition comprises from 2% to 15% by weight of (c), based on a total weight of the composition.

33. The composition of claim 20, wherein a ratio by weight of (b) and (c) is from 5:1 to 1:1.

34. The composition of claim 20, wherein the composition further comprises citric acid and/or sodium citrate.

35. The composition of claim 34, wherein a ration by weight of citric acid and sodium citrate is adjusted such that the composition has a pH of from 3.2 to 5 when the composition is dissolved in water in a ratio of 1 part composition per 20 parts of water.

36. A cosmetic product, wherein the product comprises the composition of claim 20 and packaging which encloses the composition.

37. The product of claim 36, wherein the packaging consists of a water-soluble film.

38. The product of claim 37, wherein the water-soluble film comprises or consists of one or more of polyvinyl alcohol, gelatine and alginates.

39. The product of claim 36, wherein the composition is enclosed in a paper pouch.
